(19) United States
(12) Patent Application Publication
(10) Pub. No.: US 2016/0213510 A1
(43) Pub. Date: Jul. 28, 2016

(54) THERMAL ASSISTANCE GLOVES

(71) Applicant: Qore Performance, Inc., Fairfax, VA (US)

(72) Inventors: Justin Li, Fairfax, VA (US); Jared D. Willcox, Fairfax, VA (US)

(21) Appl. No.: 15/006,954

(22) Filed: Jan. 26, 2016

Related U.S. Application Data
(60) Provisional application No. 62/108,821, filed on Jan. 28, 2015.

(51) Int. Cl. A61F 7/02; A41D 19/015; A41D 19/00

(52) U.S. Cl. CPC A61F 7/02; A41D 19/002; A41D 19/0044; A41D 19/01535; A41D 19/01541; A61F 2007/0293; A61F 2007/0236

(57) ABSTRACT

The present invention is directed to gloves which provide supplemental heating or cooling to a user's circulating arterial blood.

### CROSS REFERENCE TO RELATED APPLICATION

This application claims priority to U.S. Provisional Application Ser. No. 62/108,821, filed Jan. 28, 2015, the entirety of which is incorporated herein by reference.

### FIELD OF THE INVENTION

The present invention relates generally to gloves and more specifically to gloves which assist in regulating the blood temperature of a wearer.

### BACKGROUND

Medical personnel and First Responders alike consume large quantities of nitrile and rubber gloves in their daily duties. Infectious disease outbreaks, such as the Ebola epidemic currently underway in West Africa and select parts of Europe and the United States, compound this consumption. Nitrile and rubber gloves are used to protect the wearer from various infections and contaminants present in their work environments. Nitrile has replaced latex as the material of choice for most applications because of it is generally hypoallergenic compared to its other waterproof cousin, latex. Rubber gloves are used as an outer layer to nitrile base gloves in higher threat environments where blood borne or airborne pathogens may be present.

While nitrile, latex and rubber all provide excellent barriers to infectious disease and other biological and chemical threats, it is this very ability to defend against these threats that also creates the biggest challenge for the end-user: heat build-up. None of these materials allow for the evaporation of sweat from human skin. Evaporation is a fundamental impossibility when juxtaposed against the requirement to provide a barrier against threatening microbes. However, without evaporation, the human body is robbed of its ability to regulate its own body temperature which can lead to a host of medical problems including dehydration, heat stress/exhaustion, loss of mental acuity, heat stroke and in the worst cases, death.

Thus there remains a need for gloves which assist in regulating the body temperature of the wearer.

### SUMMARY OF THE INVENTION

The present invention provides an innovative glove configured to cool or heat circulating arterial blood of a wearer. The glove comprises a holder having a heating or cooling device, wherein the device is situated such that it is in an abiding heat transfer relation with a wearer's skin thereby allowing effective heat transfer with the wearer's circulating arterial blood.

In another aspect, the invention provides a method of regulating blood temperature. The method includes putting a glove of the invention on the hand of a user and activating the heating or cooling device thereby causing heat transfer between the heating or cooling device and circulating arterial blood of the user.

### BRIEF DESCRIPTION OF THE FIGURE

In the drawings, like elements are assigned like reference numerals. The drawings are not necessarily to scale, with the emphasis instead placed upon the principles of the present invention. Additionally, each of the embodiments depicted are but one of a number of possible arrangements utilizing the fundamental concepts of the present invention. The drawings are briefly described as follows:

FIG. 1 is a diagrammatic view of a right-handed thermal assistance glove in one embodiment of the invention.

### DETAILED DESCRIPTION

The invention relates to a glove device configured to regulate the blood temperature of a wearer. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

The present invention provides an innovative glove configured to cool or heat circulating arterial blood of a wearer. The glove comprises a holder having a heating or cooling device, wherein the device is situated such that it is in an abiding heat transfer relation with a wearer's skin thereby allowing effective heat transfer with the wearer's circulating arterial blood.

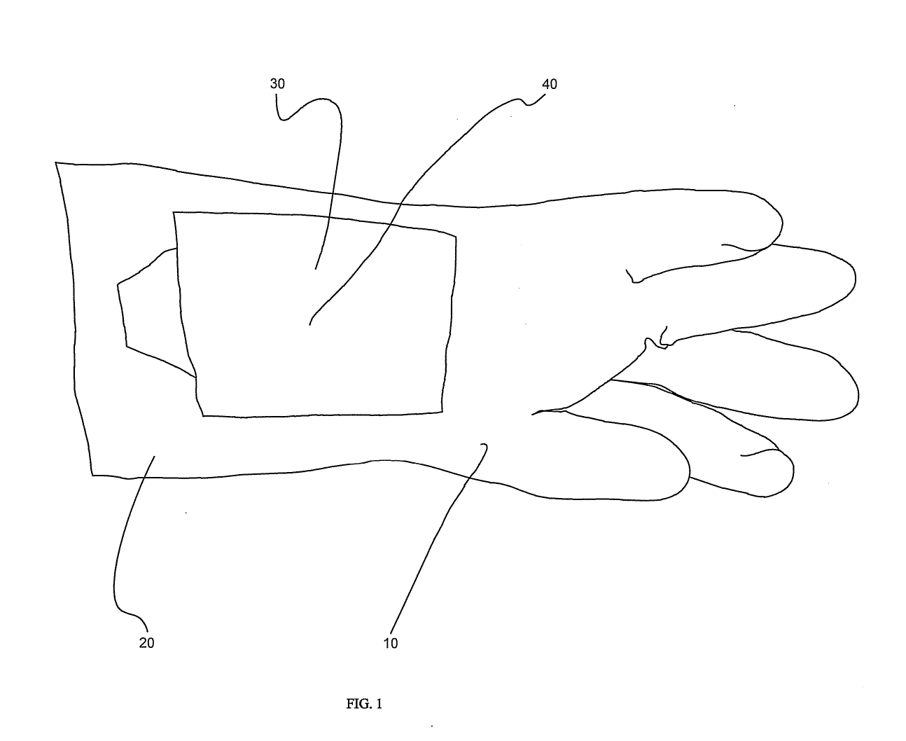

FIG. 1 illustrates one embodiment of a glove (10) of the invention. While FIG. 1 depicts a right-handed glove, one of skill in the art would understand that the invention also includes left-handed gloves. In embodiments, the glove is a gauntlet style glove having an extended cuff (20) configured to extend up the arm of a wearer. The glove includes a holder (30) which may be situated on the cuff such that in use, it is adjacent the radial or ulnar arteries. In embodiments, the cuff may extend further up the arm of the user such that the holder is situated adjacent the brachial artery. In alternative embodiments, the glove may have multiple holders such multiple arteries may be targeted.

The holder (30) is configured to receive or house a thermal device (40) configured to heat or cool circulating blood of the user. As such, the thermal device is disposed immediately adjacent to and in heat transfer relationship with the skin of the wearer. That is, the thermal device may be separated from the skin of the user by only the material of the glove. One of skill in the art would understand that heat flow may be bi-directional (i.e., extend either toward or from the thermal device) because heat transfer can be heat flow either from the thermal device to the user (i.e., supplying heat to the user), or heat flow from the user to the thermal device (i.e., cooling the user). In various embodiments, the thermal device may include reactants, which when reacted provide heat (i.e., exothermic chemical reaction) to the user, or alternatively provide cooling (i.e., endothermic chemical reaction) to the user. The thermal device may provide heating or cooling by mechanisms other than chemical reaction. For example, the thermal device may be thermoelectric, resistance, reverse Peltier effect, or any other desired form of heating or cooling.

It will be appreciated that the glove of the invention may be configured for use in a variety of environments and vocations. For example, the device may be configured for use by medical personnel, firefighters/first responders, law enforcement, military personnel, garbage collectors and the like. Additionally, the glove of the invention may be suitable for use in motorsports, military applications, construction, welding, ski/snow sports, hunting/outdoor sports, and the like. As such, the glove of the invention may be constructed of any number of natural or synthetic materials, including, but not limited to any of the following: nitrile, latex, rubber, plastic, nylon, polyester, Nomex, Carbon-X, cotton, neoprene, leather, suede, Clarino, wool, fleece, Spectra Shield, synthetic leather, canvas, Kevlar or any other material used to make gloves for any application. In one particular embodiment, the glove is constructed of pathogen resistant material such that the glove imparts resistance to infection to the user. Such a glove may be used by medical personnel treating infected patients.

In one embodiment, the glove may be a single unit construction such that the cuff or gauntlet portion of the glove extends to cover the entire arm; thereby covering multiple pulse points (ulnar/radial and brachial).

In another embodiment, the glove may be either a single or dual pulse point glove, having built-in instant cooling packs as the thermal device. As discussed above, such packs may include reactants configured to produce an endothermic reaction when reacted. For example, such packs may include various ratios of urea or ammonium nitrate and water to produce endothermic reactions when the materials are combined. One will appreciate that the built-in instant packs are not restricted to these materials, but could be any material or combination of materials which produce on-demand endothermic reactions.

Although the invention has been described with reference to the above example, it will be understood that modifications and variations are encompassed within the spirit and scope of the invention. Accordingly, the invention is limited only by the following claims.

What is claimed is:

1. A glove comprising a holder having a heating or cooling device, wherein the device is situated such that it is in an abiding heat transfer relation with a wearer's skin thereby allowing effective heat transfer with the wearer's circulating arterial blood.

2. The glove of claim 1, wherein the glove comprises a natural material, a synthetic material, or a combination thereof.

3. The glove of claim 2, wherein the glove is constructed of a pathogen resistant material.

4. The glove of claim 1, wherein the glove is a gauntlet style glove having a cuff which extends up the forearm of the wearer.

5. The glove of claim 4, wherein the holder is situated on the cuff such that it is adjacent the wearer's ulnar artery, radial artery, brachial artery, or a combination thereof.

6. The glove of claim 1, wherein the holder has a cooling device.

7. The glove of claim 6, wherein the cooling device comprises reactants configured to provide an endothermic reaction.

8. The glove of claim 1, wherein the holder has a heating device.

9. The glove of claim 8, wherein the heating device comprises reactants configured to provide an exothermic reaction.

10. The glove of claim 8, wherein the heating device comprises a mechanical heater.

11. A method of regulating blood temperature comprising putting the glove of claim 1 on the hand of a user and activating the heating or cooling device thereby causing heat transfer between the heating or cooling device and circulating arterial blood of the user.

12. The method of claim 11, wherein the glove comprises a natural material, a synthetic material, or a combination thereof.

13. The method of claim 11, wherein the glove is constructed of a pathogen resistant material.

14. The method of claim 11, wherein the glove is a gauntlet style glove having a cuff which extends up the forearm of the user.

15. The method of claim 14, wherein the holder is situated on the cuff such that it is adjacent the wearer's ulnar artery, radial artery, brachial artery, or a combination thereof.

16. The method of claim 11, wherein the holder has a cooling device.

17. The method of claim 16, wherein the cooling device comprises reactants configured to provide an endothermic reaction.

18. The method of claim 11, wherein the holder has a heating device.

19. The method of claim 18, wherein the heating device comprises reactants configured to provide an exothermic reaction.

20. The method of claim 18, wherein the heating device comprises a mechanical heater.
